(19) United States
(12) Patent Application Publication
(10) Pub. No.: US 2012/0296231 A1
(43) Pub. Date: Nov. 22, 2012

(54) BODY IMPEDANCE MEASUREMENT APPARATUS

(75) Inventors: Nobuhiko OSOEGAWA, Amagasaki-shi (JP); Masahiro KITAGAWA, Joyo-shi (JP); Tsutomu YAMASAWA, Takatsuki-shi (JP)

(73) Assignee: OMRON HEALTHCARE CO., LTD., Muko-shi (JP)

(21) Appl. No.: 13/565,548

(22) Filed: Aug. 2, 2012

Related U.S. Application Data
(63) Continuation of application No. PCT/JP2011/050349, filed on Jan. 12, 2011.

(30) Foreign Application Priority Data
Mar. 23, 2010 (JP) 2010-066327

(51) Int. Cl. A61B 5/053

(52) U.S. Cl. 600/547

(57) ABSTRACT

A body impedance measurement apparatus having high aesthetic qualities can be realized without increasing the manufacturing costs thereof by providing a membrane-type electrode formed integrally with the surface of a resinous first housing through insert molding.

This is a Continuation of Application No. PCT/JP2011/050349 filed Jan. 12, 2011. The disclosure of the prior application is hereby incorporated by reference herein in its entirety.

### TECHNICAL FIELD

This invention relates to body impedance measurement apparatuses that measure the impedance of a body.

### BACKGROUND ART

JP 2006-230700A (Patent Literature 1) and JP 2007-117624A (Patent Literature 2) can be given as examples of body impedance measurement apparatuses that measure the impedance of a body. In these body impedance measurement apparatuses, electrodes employing a metal plate are brought into contact with a body in order to apply a current to and measure a voltage from the body.

However, in such body impedance measurement apparatuses, electrodes having a large surface area so as to handle hands and feet of different sizes for different users are exposed on the exterior of the body impedance measurement apparatus, which greatly influences the aesthetics of the body impedance measurement apparatus.

Although it is possible to employ a complex shape with a high aesthetic quality for the electrodes themselves, this leads to an increase in the cost of the body impedance measurement apparatus. Furthermore, because the hands and feet of the user come into direct contact with the metal plate used for the electrodes, there are cases where the body impedance measurement apparatus feels cold to the user.

Meanwhile, the body impedance measurement apparatus disclosed in JP 2002-172100A (Patent Literature 3) employs a resinous cover, in which an insulating resin and a conductive resin are formed in an integrated manner, as the housing of the body impedance measurement apparatus.

However, such a body impedance measurement apparatus uses a conductive resin, which is a special type of resin material, and thus there are limits to grades that can be selected for the conductive resin material if a desired shapability, color, and so on are to be obtained. Furthermore, dedicated metal molding equipment (for integrated multi-color molding) is required for the conductive resin material, which increases the equipment costs and the like; this in turn leads to an increase in the cost of the body impedance measurement apparatus.

Using a glass housing and adding a transparent electrode film to the surface of the glass housing to serve as the electrodes can be considered as another structure. However, it is necessary for the surface of the glass housing to be made flat in order to manufacture the glass housing at a cost where mass production is possible, and thus a complex shape with a high aesthetic quality cannot be employed.

Meanwhile, the conduction terminals between the electrodes and the internal wiring are disposed on the upper surfaces of the electrodes, and thus the component serving as the cover of the conduction terminals is shaped so as to protrude further from the surface than the surfaces of the electrodes; this again greatly influences the aesthetics of the body impedance measurement apparatus. There are also situations where this protrusion contacts the hands and feet of the user and thus interferes with the user when the hands and feet of the user come into direct contact with the electrode surface.

### CITATION LIST

### Patent Literature

Patent Literature 1: JP 2006-230700A
Patent Literature 2: JP 2007-117624A
Patent Literature 3: JP 2002-172100A

### SUMMARY OF INVENTION

### Technical Problem

Problems to be solved by this invention include the aesthetics of the body impedance measurement apparatus being greatly influenced and the body impedance measurement apparatus feeling cold to the user in the case where electrodes that use a metal plate are employed in the body impedance measurement apparatus. A further problem is an increase in the cost of the body impedance measurement apparatus in the case where a special material such as a conductive resin material, glass, or the like is used in the housing of the body impedance measurement apparatus.

Having been achieved to solve the stated problems, it is an object of this invention to provide a body impedance measurement apparatus that enables a shape that has a high aesthetic quality to be employed in the body impedance measurement apparatus and that has a structure that does not lead to an increase in the cost of the body impedance measurement apparatus.

### Solution to Problem

According to one aspect of this invention, a body impedance measurement apparatus is an impedance measurement apparatus that measures the impedance of a body, and includes a housing in which an electronic component is contained and an electrode, provided on a surface of the housing, that makes contact with the body when measuring the impedance of the body; a base portion of the housing on which the electrode is provided is a resin molded component; and the electrode is a membrane-type electrode.

In the stated invention, it is preferable for the membrane-type electrode to be a transparent conductive membrane or a web-form conductive membrane.

In the stated invention, it is preferable for the membrane-type electrode to be formed on a film.

In the stated invention, it is preferable for the film on which the membrane-type electrode is formed to be integrated with the surface of the housing through insert molding when the resin of the housing is molded.

In the stated invention, it is preferable for the membrane-type electrode to be an electrode membrane formed on the surface of the housing through sputtering.

In the stated invention, it is preferable for the membrane-type electrode to be an electrode membrane formed on the surface of the housing through coating.

In the stated invention, it is preferable for the membrane-type electrode to be an electrode membrane formed on the surface of the housing through printing.

In the stated invention, it is preferable for the apparatus to further include a recessed region provided on the surface of the housing and a cap that covers the recessed region; and for the membrane-type electrode to include an extended region provided in the recessed region, and for the extended region to be conductive, at the recessed region, with the electronic component contained within the housing.

In the stated invention, it is preferable for the housing to include a first housing and a second housing, the membrane-type electrode to be provided on a surface of the first housing, the membrane-type electrode to include an extended region that wraps around to the rear surface side of the first housing, and the extended region to be conductive, at the rear surface side of the first housing, with the electronic component contained within the housing.

According to another aspect of this invention, a body impedance measurement apparatus is an impedance measurement apparatus that measures the impedance of a body, and includes a housing in which an electronic component is contained and an electrode, provided on a surface of the housing, that makes contact with the body when measuring the impedance of the body; the surface of the housing on which the electrode is provided has a curved surface portion, and the electrode is a membrane-type electrode.

### Advantageous Effects of Invention

According to a body impedance measurement apparatus based on this invention, it is possible to provide a body impedance measurement apparatus in which it is easy to employ a shape having high aesthetic qualities for the body impedance measurement apparatus, and that has a structure that does not increase the cost of the body impedance measurement apparatus.

### BRIEF DESCRIPTION OF DRAWINGS

FIG. 1 is a first perspective view illustrating the external configuration of a body impedance measurement apparatus according to a first embodiment.

FIG. 2A, FIG. 2B and FIG. 2C are a plan view, a front view, and a right-side view, respectively, illustrating the external configuration of the body impedance measurement apparatus according to the first embodiment.

FIG. 3 is a diagram illustrating a measurement posture taken when a user uses the body impedance measurement apparatus according to the first embodiment.

FIG. 4 is a block diagram of the body impedance measurement apparatus according to the first embodiment.

FIG. 5 is a second perspective view illustrating the external configuration of the body impedance measurement apparatus according to the first embodiment.

FIG. 6 is a cross-sectional view taken along the VI-VI arrows in FIG. 2A.

FIG. 7 is an enlarged partial perspective view illustrating the region enclosed by VII in FIG. 5.

FIG. 8 is an enlarged partial cross-sectional view illustrating the region enclosed by VIII in FIG. 6.

FIG. 9 is an enlarged partial perspective view illustrating an area that corresponds to the region enclosed by VII in FIG. 5, in another body impedance measurement apparatus according to the first embodiment.

FIG. 10 is a first perspective view illustrating the external configuration of a body impedance measurement apparatus according to a second embodiment.

FIG. 11A, FIG. 11B and FIG. 11C are a plan view, a front view, and a right-side view, respectively, illustrating the external configuration of the body impedance measurement apparatus according to the second embodiment.

FIG. 12 is a second perspective view illustrating the external configuration of the body impedance measurement apparatus according to the second embodiment.

FIG. 13 is a cross-sectional view taken along the XIII-XIII arrows in FIG. 11A.

FIG. 14 is an enlarged partial perspective view illustrating the region enclosed by XIV in FIG. 12.

FIG. 15 is an enlarged partial cross-sectional view illustrating the region enclosed by XV in FIG. 13.

FIG. 16 is a first perspective view illustrating the external configuration of a body impedance measurement apparatus according to a third embodiment.

FIG. 17A, FIG. 17B and FIG. 17C are a plan view, a front view, and a right-side view, respectively, illustrating the external configuration of the body impedance measurement apparatus according to the third embodiment.

FIG. 18 is a second perspective view illustrating the external configuration of the body impedance measurement apparatus according to the third embodiment.

FIG. 19 is an enlarged partial perspective view illustrating the region enclosed by XIX in FIG. 18.

FIG. 20 is a cross-sectional view taken along the XX-XX arrows in FIG. 17A.

FIG. 21 is an enlarged partial cross-sectional view illustrating the region enclosed by XXI in FIG. 20.

FIG. 22 is a first perspective view illustrating the external configuration of a body impedance measurement apparatus according to a fourth embodiment.

FIGS. 23A, 23B, and 23C are a plan view, a front view, and a right-side view, respectively, illustrating the external configuration of the body impedance measurement apparatus according to the fourth embodiment.

FIG. 24 is a diagram illustrating a measurement posture taken when a user uses the body impedance measurement apparatus according to the fourth embodiment.

FIG. 25 is a block diagram of the body impedance measurement apparatus according to the fourth embodiment.

FIG. 26 is a second perspective view illustrating the external configuration of the body impedance measurement apparatus according to the fourth embodiment.

FIG. 27 is an enlarged partial perspective view illustrating the region enclosed by XXVII in FIG. 26.

FIG. 28 is an enlarged partial perspective view illustrating a state of conductivity between a membrane-type electrode and an electronic component in the body impedance measurement apparatus according to the fourth embodiment.

### DESCRIPTION OF EMBODIMENTS

Hereinafter, body impedance measurement apparatuses according to respective embodiments of this invention will be described in detail with reference to the drawings. When numbers, amounts, and so on are discussed in the following embodiments, it should be noted that unless explicitly mentioned otherwise, the scope of the present invention is not necessarily limited to those numbers, amounts, and so on. Furthermore, in the case where multiple embodiments are given hereinafter, it is assumed from the outset that the configurations of the respective embodiments can be combined as appropriate unless explicitly mentioned otherwise. In the drawings, identical reference numerals refer to identical or corresponding elements; there are also cases where redundant descriptions are omitted.

### First Embodiment

### Body Impedance Measurement Apparatus 100

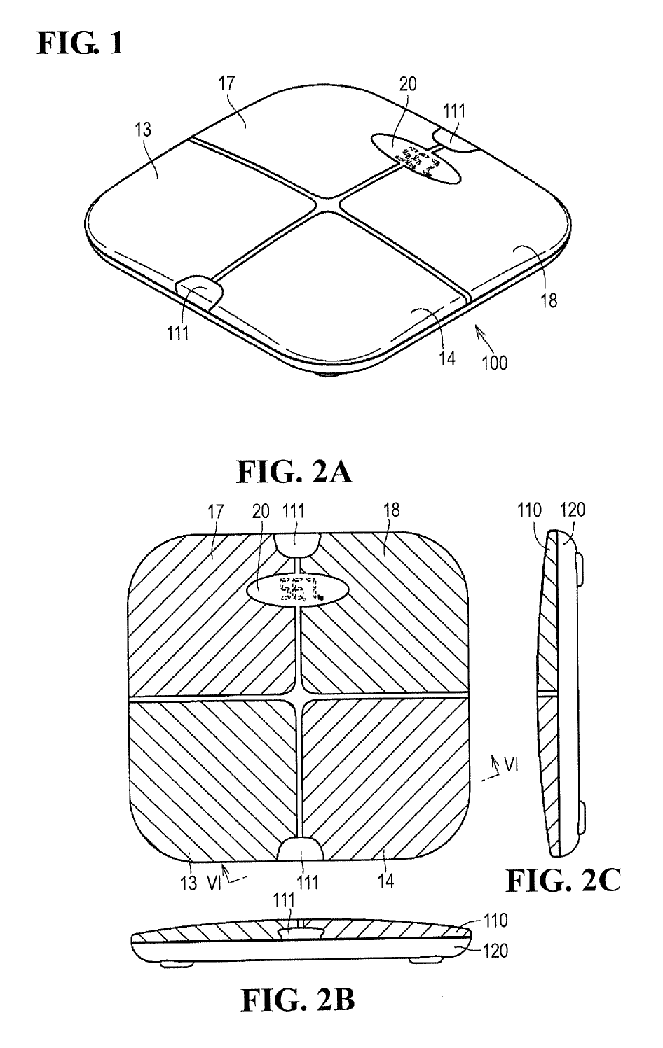

Hereinafter, a body impedance measurement apparatus 100 according to a first embodiment will be described with reference to FIGS. 1 through 9. First, the general configuration of the body impedance measurement apparatus 100 will be described with reference to FIGS. 1 through 5.

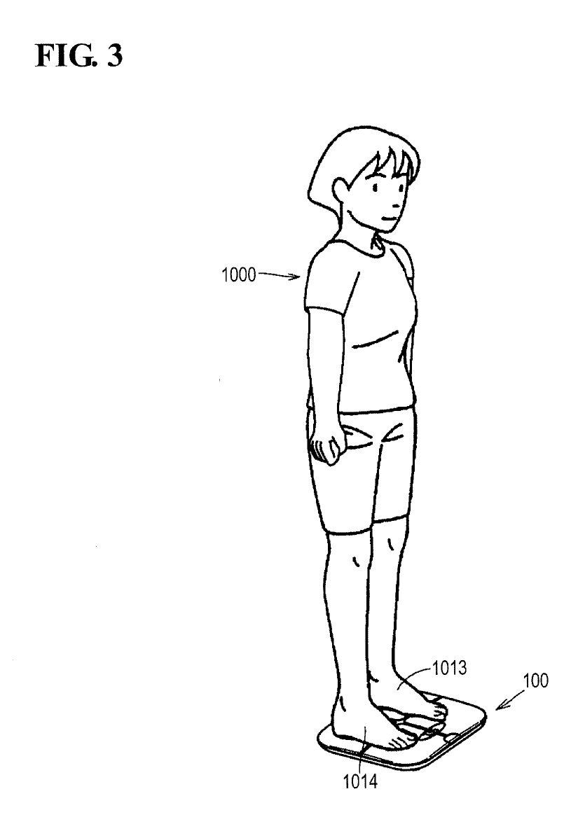

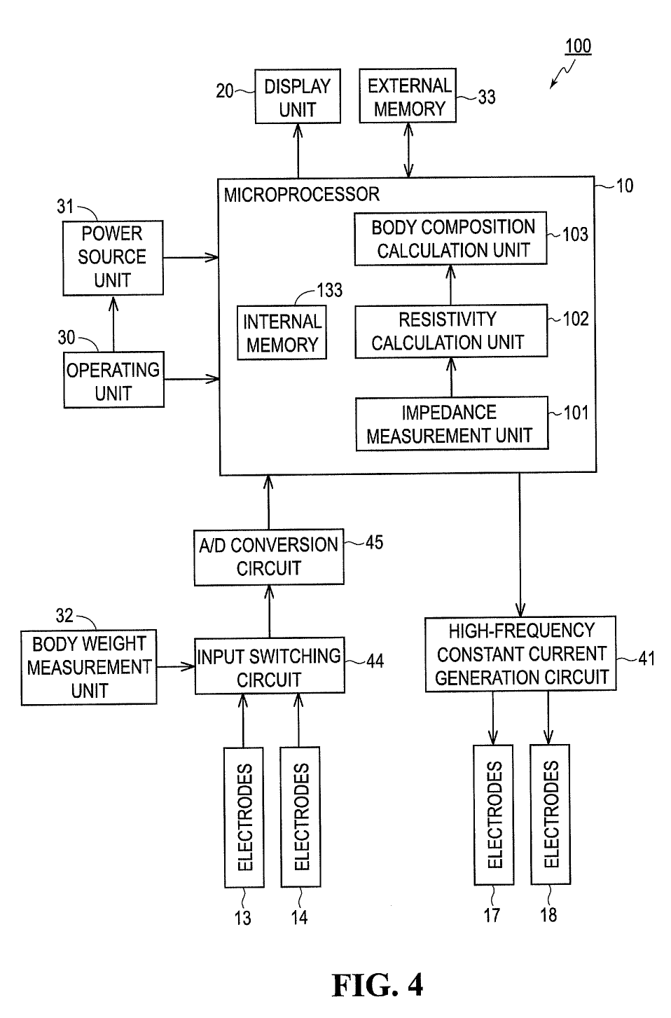

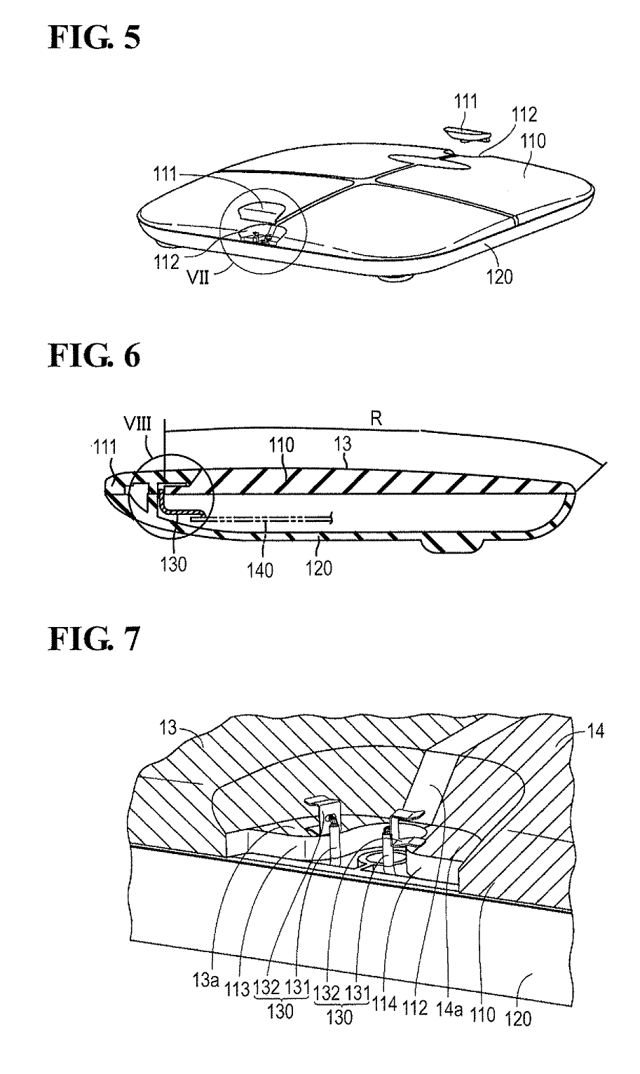

Note that FIG. 1 is a first perspective view illustrating the external configuration of the body impedance measurement apparatus 100, FIGS. 2A, 2B, and 2C are a plan view, a front view, and a right-side view, respectively, illustrating the external configuration of the body impedance measurement apparatus 100, FIG. 3 is a diagram illustrating a measurement posture taken when a user uses the body impedance measurement apparatus 100, FIG. 4 is a block diagram of the body impedance measurement apparatus 100, and FIG. 5 is a second perspective view illustrating the external configuration of the body impedance measurement apparatus 100.

The body impedance measurement apparatus 100 includes a first housing 110 located on a top surface side and a second housing 120 located on a bottom surface side. The first housing 110 and the second housing 120 have, when viewed from above, rectangular shapes whose corners are formed so as to be rounded. Meanwhile, as can be seen in FIGS. 2A, 2B, and 2C, the surface of the first housing 110 has a form that is curved so as to bulge upward.

A display unit 20 is provided in the surface of the first housing 110. For example, a liquid-crystal display device (LCD) or the like is used for the display unit 20. In addition, electrodes 13, 14, 17, and 18 are provided on the surface of the first housing 110 so as to divide that surface into four sections. Membrane-type electrodes are used for the electrodes 13, 14, 17, and 18 in the present embodiment. The electrodes 13, 14, 17, and 18 are provided in the hatched regions shown in FIGS. 2A, 2B, and 2C.

The electrode 13 located in the lower-left area of the body impedance measurement apparatus 100 is an electrode for measuring a voltage during impedance measurement, whereas the electrode 17 located in the upper-left area is an electrode for applying a current during impedance measurement. The electrodes 13 and 17 make contact with the sole of the user's left foot.

The electrode 14 located in the lower-right area of the body impedance measurement apparatus 100 is an electrode for measuring a voltage during impedance measurement, whereas the electrode 18 located in the upper-right area is an electrode for applying a current during impedance measurement. The electrodes 14 and 18 make contact with the sole of the user's right foot.

Removable caps 111 are provided in two locations in the peripheral edges of the first housing 110. The surfaces of the caps 111 are formed so as to be flush with the surface of the first housing 110. The purpose of the caps 111 will be described later.

In the present embodiment, membrane-type electrodes, in which an electrode membrane is formed on a transparent film, are used. Meanwhile, the film on which the membrane-type electrodes are formed is integrated with the surface of the first housing 110 through insert molding during the resin molding of the first housing 110. The transparency of the transparent film is not particularly an issue as long as the color of the resin used for the first housing 110 can be seen therethrough. It is also possible to use an opaque film.

An acrylic resin (for example, a polymethyl methacrylate resin (PMMA)), polycarbonate, acrylonitrile butadiene styrene resin (ABS resin), or the like is used for the material of the first housing 110. Although basically the same material as the material of the first housing 110 is used for the material of the second housing 120, it should be noted that, depending on the application, another material may be used instead.

ITO (indium tin oxide), ZnO (zinc oxide), Ag (silver) ink, a conductive high-polymer (a polyacetylene, a polythiophene, a polyethylenedioxythiophene, or the like) is used as the material of the electrode membrane. The electrode membrane is approximately 10 nm to 1 μm thick.

Polyethlene terephthalate (PET), polyimide, or the like is used as the material of the film. The film is approximately 10 μm to 500 μm thick.

The injection molding conditions for the insert molding are, when for example a polymethyl methacrylate resin (PMMA) is used as the material for the first housing 110, a resin temperature of approximately 200° C. to 270° C., an injection pressure of approximately 60 MPa to 140 MPa, and a mold temperature of approximately 40° C. to 80° C. Note that the optimum conditions for the heat resistance, molded component shapes, and other factors of the film used in the membrane-type electrodes are determined as appropriate.

Note also that the membrane-type electrodes are not limited to a transparent conductive membrane, and a web-form conductive membrane can also be used. Furthermore, the method for integrating the film is not limited to insert molding, and a method in which an electrode membrane is formed on the surface of the first housing 110 through sputtering, a method in which an electrode membrane is formed on the surface of the first housing 110 through coating, forming an electrode membrane on the surface of the first housing 110 through printing, and so on can also be used for the electrodes.

As shown in FIG. 3, when the body impedance measurement apparatus 100 is being used, the body impedance measurement apparatus 100 is placed on a flat placement surface, and a user 1000 stands upon the body impedance measurement apparatus 100. The impedance of the user is measured by a left foot 1013 of the user making contact with the electrodes 13 and 17 and a right foot 1014 of the user making contact with the electrodes 14 and 18.

As shown in the block diagram in FIG. 4, the body impedance measurement apparatus 100 includes: the aforementioned multiple electrodes 13, 14, 17, and 18; the display unit 20; an operating unit 30; a body weight measurement unit 32; and a microcomputer 10 for carrying out the overall control of the body impedance measurement apparatus 100, processes such as various types of computations, and so on.

Furthermore, a high-frequency constant current generation circuit 41 that generates a high-frequency constant current of a predetermined frequency, an input switching circuit 44 for switching an input to either voltage information obtained from the electrodes 17 and 18 used for current application and the electrodes 13 and 14 used for voltage measurement or body weight information obtained from the body weight measurement unit 32, and an analog (A)/digital (D) conversion circuit 45 for converting the voltage information and the body weight information obtained from the input switching circuit 44 from analog signals into digital signals are also included.

Furthermore, a power source unit 31 for supplying electrical power to the microcomputer 10 when a power switch included in the operating unit 30 is manipulated and an external memory 33 for storing information such as measurement results are also included.

In addition, the microcomputer 10 includes an internal memory 133, such as an EEPROM (Electrically Erasable Programmable Read-Only Memory), used for storing various types of control programs and the like. The microcomputer 10 includes an impedance measurement unit 101, a resistivity calculation unit 102, and a body composition calculation unit 103, and measures an impedance, calculates a resistivity, and calculates a body composition in accordance with programs stored in the internal memory 133.

In addition, the microcomputer 10 measures a body weight through a known method based on a signal from the body weight measurement unit 32, which is for example a body weight sensor, obtained via the A/D conversion circuit 45. Furthermore, the microcomputer 10 generates signals for displaying measurement results and the like obtained by the body composition calculation unit 103 in the display unit 20. The microcomputer 10 also writes and reads to and from the external memory 33.

Fat mass, fat-free mass, muscle mass, bone mass, body fat percentage, muscle percentage, visceral fat levels, and so on can be given as examples of body compositions that can be measured by the body impedance measurement apparatus 100 according to the present embodiment. These body compositions are all calculated by the body composition calculation unit 103 through known methods based on body impedance values obtained by the stated impedance measurement unit 101 and personal data of the user such as his/her height, weight, age, sex, and so on recorded in the internal memory.

Conduction Structure

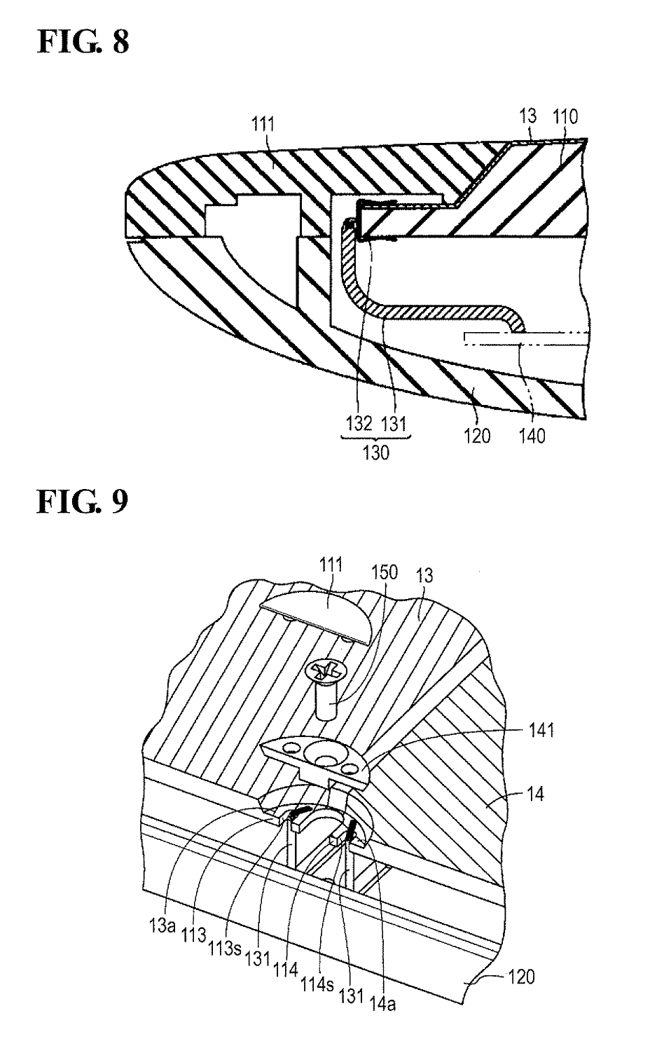

Next, a conduction structure between an electronic component 140 housed between the first housing 110 and the second housing 120 and the electrodes 13, 14, 17, and 18 provided on the surface of the first housing 110 will be described with reference to FIGS. 5 through 8. Note that FIG. 5 is a second perspective view illustrating the external configuration of the body impedance measurement apparatus 100, FIG. 6 is a cross-sectional view taken along the VI-VI arrows in FIG. 2A, FIG. 7 is an enlarged partial perspective view illustrating the region enclosed by VII in FIG. 5, and FIG. 8 is an enlarged partial cross-sectional view illustrating the region enclosed by VIII in FIG. 6.

As shown in FIG. 5, recessed regions 112 for securing a conduction structure between the electronic component 140 housed within the first housing 110 and the second housing 120 and the electrodes 13, 14, 17, and 18 provided on the surface of the first housing 110 are provided in two locations at the perhiperal edges of the surface of the first housing 110, and the removable caps 111 are provided so as to cover the respective recessed regions 112.

Because the recessed regions 112 in the two locations have the same structure, the conduction structure in the region enclosed by VII in FIG. 5 will be described with reference to FIGS. 6 through 8. The half-circle shaped recessed region 112 is formed in the surface of the first housing 110 so as to straddle the electrode 13 and the electrode 14. Protruding regions 113 and 114 that define a base surface are formed in the recessed region 112.

An extended region 13a of the electrode 13 is formed so as to extend to the protruding region 113 of the recessed region 112, and an extended region 14a of the electrode 14 is formed so as to extend to the protruding region 114. As shown in FIG. 6, the electrodes 13, 14, 17, and 18 are respectively formed on the surface of the first housing 110 from the ends of the peripheral edges to the protruding regions 113 and 114 of the recessed region 112 (the range indicated by R).

The electronic component 140 is provided with connectors 130 for conductivity with the extended regions 13a and 14a. Each of the connectors 130 includes a cable 131 and a clip 132.

As shown in FIG. 8, in the recessed region 112, the clip 132 of the connector 130 fits around the protruding region 113 of the first housing 110, which electrically connects the clip 132 and the extended region 13a. This conduction structure is the same for the electrode 14, the electrode 17, and the electrode 18.

A known engagement structure (not shown) is employed between the cap 111 and the recessed region 112; the cap 111 is normally held in an engaged state with the recessed region 112 but can be removed from the recessed region 112 by imparting an external force thereupon.

Note that FIG. 9 illustrates another conduction structure. Slits 113s and 114s are provided in the protruding regions 113 and 114, respectively, and the cables 131 are fitted into the slits 113s and 114s; this makes it possible to achieve conductivity between the extended region 13a and the cable 131 and between the extended region 14a and the cable 131. In this case, a plate 141 is anchored to the second housing 120 using a screw 150 in order to anchor the cables 131 and 131. The plate 141 and the screw 150 are then hidden by the cap 111. The surface of the cap 111 is formed so as to be flush with the surface of the first housing 110.

Actions and Effects

According to the body impedance measurement apparatus 100 of the present embodiment described thus far, providing the membrane-type electrodes on the surface of the resinous housing makes it possible to employ a shape with high aesthetic qualities without inducing an increase in manufacturing costs.

Furthermore, because a normally-used resin can be used for the resin of the housing, the desired grade of shapability can be selected more easily than when a conductive resin, which is a special material, is used; thus existing knowledge and experience can be applied in response to many problems that occur in resin housings with ease, such as sink marks, welding lines, and so on.

In addition, even in the case where a film on which a membrane-type electrode has been formed is used, the film need only be inserted into the mold, and thus the mold structure can be made simpler, and a rise in manufacturing costs can be suppressed more, than in the case where multicolor molding or the like is employed. Furthermore, it is easy to introduce film due to its selectability from among commercial products, which also makes it possible to suppress a rise in manufacturing costs.

Furthermore, complex three-dimensional shapes, which are difficult with metal electrodes and glass housings, can be realized with ease when forming the housing from a resin, and thus a sure state of contact can be made by forming the surface of the membrane-type electrodes in the body impedance measurement apparatus 100 that make contact with the body in a shape that conforms to the contours of human feet. And although there are slight differences from person to person, the likelihood of the electrodes feeling cold to the user can be suppressed.

In addition, using a transparent film for the membrane-type electrodes allows the color of the first housing to appear on the surface, which in turn makes it possible to easily increase the color variations of the body impedance measurement apparatus 100. Furthermore, using a transparent resin such as an acryl for the housing makes it possible to make the entire housing transparent.

In addition, in the case where the electrodes are formed in an integrated manner with the housing insert molding or the like, it is easy to form the electrodes in a three-dimensional manner as compared to a case in which membrane-type electrodes are affixed using an adhesive or the like; this also makes it possible to reduce the chance of problems such as the electrodes peeling away or the like.

Furthermore, even though the recessed regions 112 are provided in the surface of the housing, the extended regions 13a and 14a of the electrodes 13 and 14 are formed so as to extend to the protruding regions 113 and 114 that define the base surfaces of the recessed regions 112, and it is therefore possible to realize a conduction structure with the electronic component 140 with ease. In addition, covering the recessed regions 112 with the caps 111 makes it possible to achieve a surface that is flush with the surface of the housing, which in turn makes it possible to improve the aesthetic qualities of the body impedance measurement apparatus 100.

Furthermore, in the case where the membrane-type electrodes are formed through sputtering, coating, or the like, the electrodes can be formed directly on the surface of the housing, which makes it possible to reduce the number of components; this in turn makes it possible to suppress a rise in the manufacturing costs.

### Second Embodiment

### Body Impedance Measurement Apparatus 200

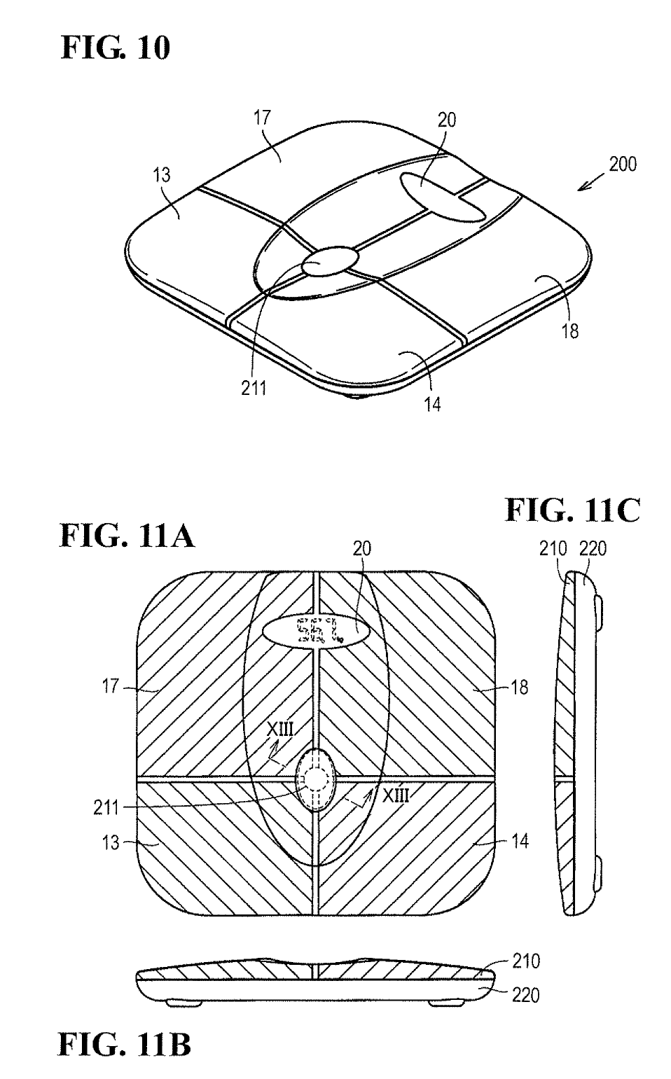

Next, a body impedance measurement apparatus 200 according to a second embodiment will be described with reference to FIGS. 10 through 15. First, the general configuration of the body impedance measurement apparatus 200 will be described with reference to FIGS. 10 and 11.

Note that FIG. 10 is a first perspective view illustrating the external configuration of the body impedance measurement apparatus 200, and FIGS. 11A, 11B, and 11C are a plan view, a front view, and a right-side view, respectively, illustrating the external configuration of the body impedance measurement apparatus 200.

The basic configuration of the body impedance measurement apparatus 200 according to the present embodiment is the same as that of the body impedance measurement apparatus 100 according to the aforementioned first embodiment; the differences lie in the shape of the surface and the location in which the conduction structure is provided. Here, the difference in the location in which the conduction structure is provided will be described in detail.

The body impedance measurement apparatus 200 includes a first housing 210 located on a top surface side and a second housing 220 located on a bottom surface side. The first housing 210 and the second housing 220 have, when viewed from above, rectangular shapes whose corners are formed so as to be rounded. Meanwhile, as can be seen in FIGS. 10, 11A, 11B, and 11C, the surface of the first housing 210 has a form that is curved so as to bulge upward, whereas the central area thereof has an oval-shaped recess.

The display unit 20 is provided in the surface of the first housing 210. In addition, the electrodes 13, 14, 17, and 18 are provided on the surface of the first housing 210 so as to divide that surface into four sections. The electrodes 13, 14, 17, and 18 are provided in the hatched regions shown in FIGS. 11A, 11B, and 11C.

Note that the same membrane-type electrodes as those used in the body impedance measurement apparatus 100 according to the first embodiment are used as the electrodes 13, 14, 17, and 18 in the present embodiment. In addition, the same material as that used in the body impedance measurement apparatus 100 according to the first embodiment is used in the first housing 210 and the second housing 220.

Conduction Structure

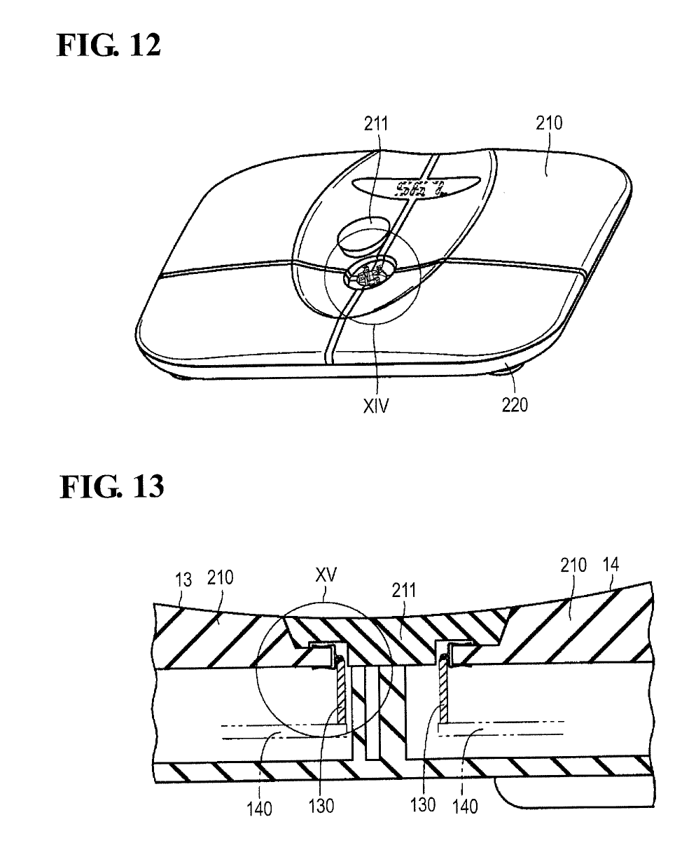

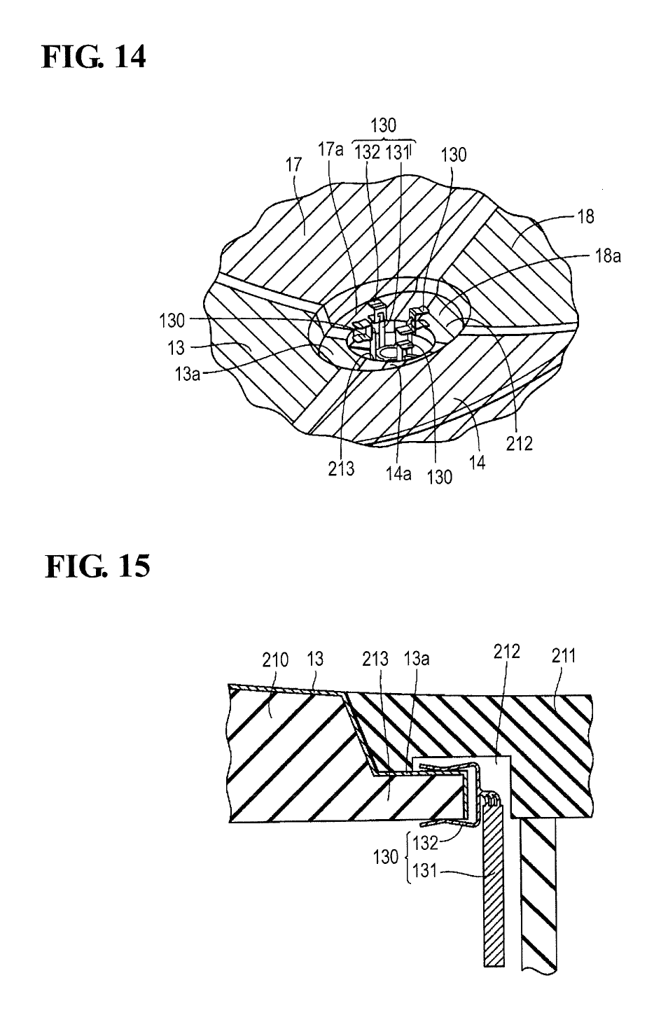

Next, a conduction structure between the electronic component 140 housed between the first housing 210 and the second housing 220 and the electrodes 13, 14, 17, and 18 provided on the surface of the first housing 210 will be described with reference to FIGS. 12 through 15. Note that FIG. 12 is a second perspective view illustrating the external configuration of the body impedance measurement apparatus 200, FIG. 13 is a cross-sectional view taken along the XIII-XIII arrows in FIG. 11A, FIG. 14 is an enlarged partial perspective view illustrating the region enclosed by XIV in FIG. 12, and FIG. 15 is an enlarged partial cross-sectional view illustrating the region enclosed by XV in FIG. 13.

As shown in FIG. 12, a recessed region 212 for securing a conduction structure between the electronic component 140 housed within the first housing 210 and the second housing 220 and the electrodes 13, 14, 17, and 18 provided on the surface of the first housing 210 is provided in one location in what is essentially the central region (that is, a region in the vicinity of the electrodes 13, 14, 17, and 18) of the surface of the first housing 210; a removable cap 211 is provided so as to cover the recessed region 212.

As shown in FIGS. 13 through 15, the circular recessed region 212 is formed in the surface of the first housing 210 so as to span the electrodes 13, 14, 17, and 18. A protruding region 213 having an open region in the center thereof is formed in the recessed region 212.

An extended region 13a of the electrode 13, an extended region 14a of the electrode 14, an extended region 17a of the electrode 17, and an extended region 18a of the electrode 18 are formed so as to extend to the protruding region 213 of the recessed region 212.

The electronic component 140 is provided with connectors 130 for conductivity with the extended regions 13a, 14a, 17a, and 18a. Each of the connectors 130 includes a cable 131 and a clip 132.

As shown in FIG. 15, in the recessed region 212, the clip 132 of the connector 130 fits around the protruding region 213 of the first housing 210, which electrically connects the clip 132 and the extended region 13a. This conduction structure is the same for the electrode 14, the electrode 17, and the electrode 18.

A known engagement structure (not shown) is employed between the cap 211 and the recessed region 212; the cap 211 is normally held in an engaged state with the recessed region 212 but can be removed from the recessed region 212 by imparting an external force thereupon.

Actions and Effects

The same actions and effects as the body impedance measurement apparatus 100 according to the aforementioned first embodiment can be achieved by the body impedance measurement apparatus 200 according to the present embodiment. In addition, by providing the recessed region 212 in the central region of the surface of the first housing 210, operations for connecting the clips 132 to the extended regions 13a, 14a, 17a, and 18a need only be carried out at one location, which makes it possible to improve the workability during the assembly process.

### Third Embodiment

### Body Impedance Measurement Apparatus 300

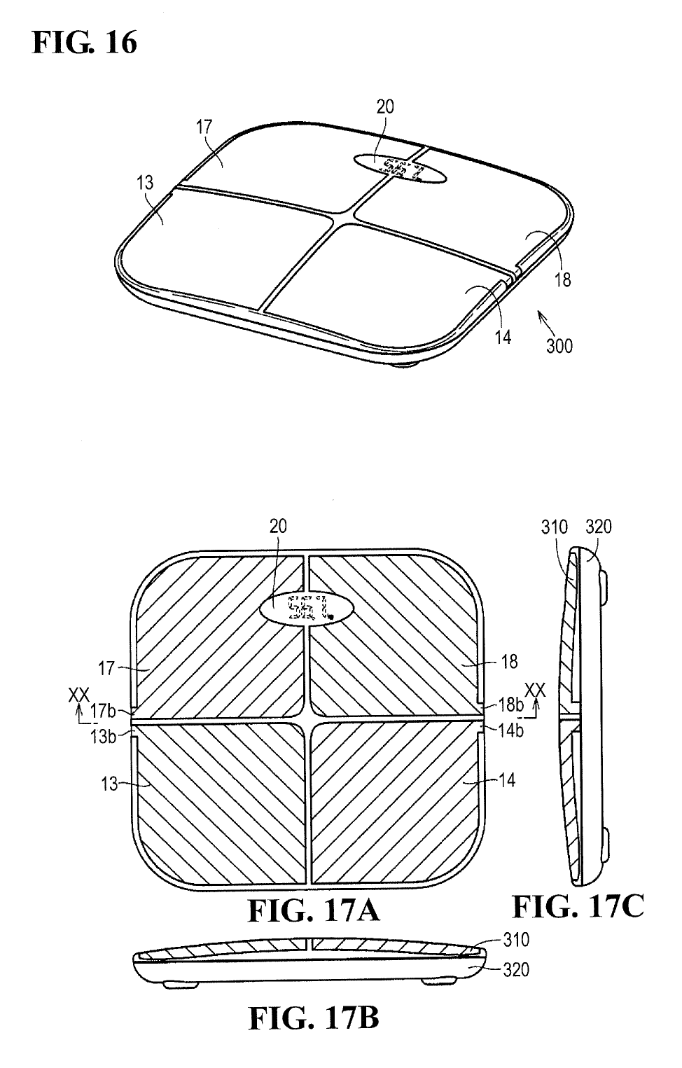

Next, a body impedance measurement apparatus 300 according to a third embodiment will be described with reference to FIGS. 16 through 21. First, the general configuration of the body impedance measurement apparatus 300 will be described with reference to FIGS. 16 and 17.

Note that FIG. 16 is a first perspective view illustrating the external configuration of the body impedance measurement apparatus 300, and FIGS. 17A, 17B, and 17C are a plan view, a front view, and a right-side view, respectively, illustrating the external configuration of the body impedance measurement apparatus 300.

The basic configuration of the body impedance measurement apparatus 300 according to the present embodiment is the same as that of the body impedance measurement apparatus 100 according to the aforementioned first embodiment; the differences lie in the shape of the surface and the location in which the conduction structure is provided. Here, the difference in the location in which the conduction structure is provided will be described in detail.

The body impedance measurement apparatus 300 includes a first housing 310 located on a top surface side and a second housing 320 located on a bottom surface side. The first housing 310 and the second housing 320 have, when viewed from above, rectangular shapes whose corners are formed so as to be rounded. Meanwhile, as can be seen in FIGS. 16, 17A, 17B, and 17C, the surface of the first housing 310 has a form that is curved so as to bulge upward.

The display unit 20 is provided in the surface of the first housing 310. In addition, the electrodes 13, 14, 17, and 18 are provided on the surface of the first housing 310 so as to divide that surface into four sections. The electrodes 13, 14, 17, and 18 are provided in the hatched regions shown in FIGS. 17A, 17B, and 17C.

Note that the same membrane-type electrodes as those used in the body impedance measurement apparatus 100 according to the first embodiment are used as the electrodes 13, 14, 17, and 18 in the present embodiment. In addition, the same material as that used in the body impedance measurement apparatus 100 according to the first embodiment is used in the first housing 310 and the second housing 320.

Conduction Structure

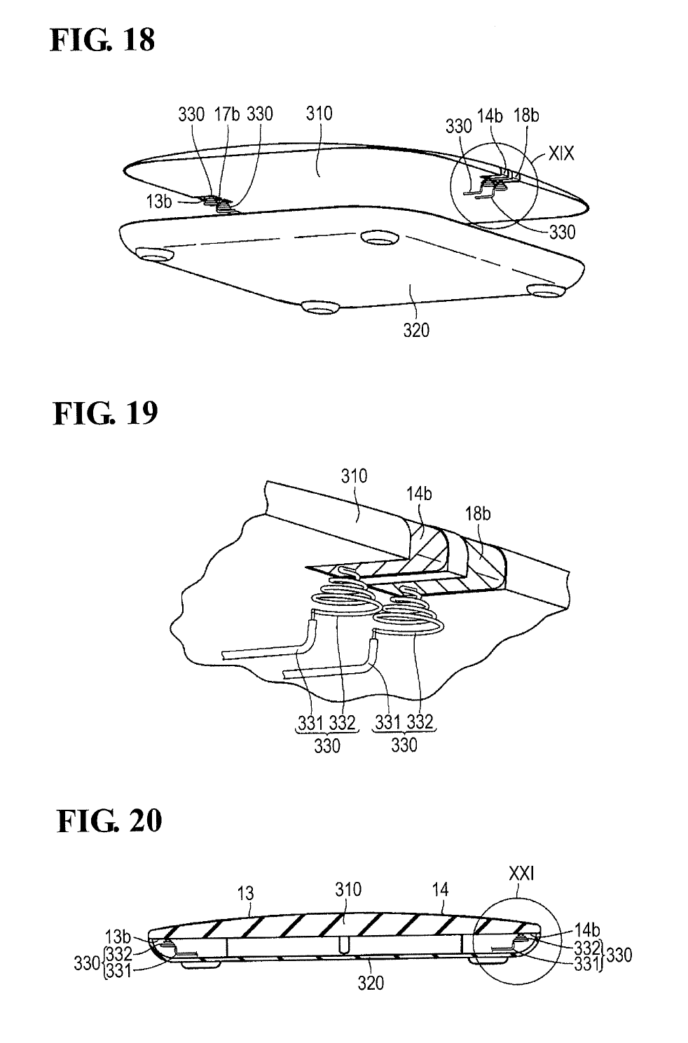

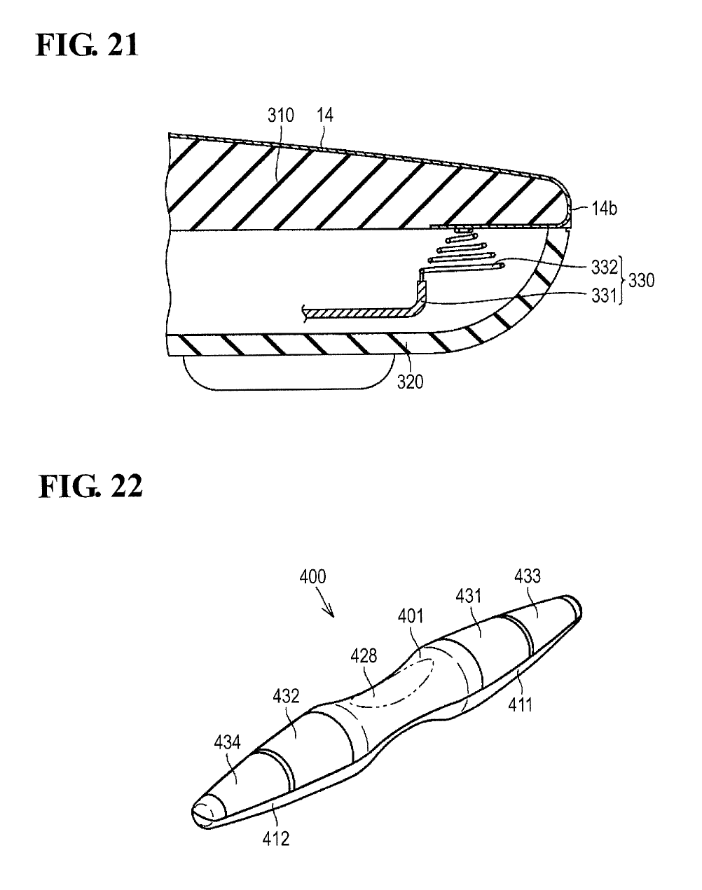

Next, a conduction structure between the electronic component 140 housed between the first housing 310 and the second housing 320 and the electrodes 13, 14, 17, and 18 provided on the surface of the first housing 310 will be described with reference to FIGS. 17 through 21. Note that FIG. 18 is a second perspective view illustrating the external configuration of the body impedance measurement apparatus 300, FIG. 19 is an enlarged partial perspective view illustrating the region enclosed by XIX in FIG. 18, FIG. 20 is a cross-sectional view taken along the XX-XX arrows in FIG. 17A, and FIG. 21 is an enlarged partial cross-sectional view illustrating the region enclosed by XXI in FIG. 20.

As shown in FIGS. 17A, 17B, and 17C, extended regions 13b, 14b, 17b, and 18b, in which the electrodes 13, 14, 17, and 18, respectively, wrap around to the rear surface side of the first housing 310, are provided in the surface of center regions of the peripheral edges of the first housing 310. The extended region 13b and the extended region 17b are disposed in the vicinity of each other, and the extended region 14b and the extended region 18b are disposed in the vicinity of each other.

The electronic component 140 is provided with connectors 330 for conductivity with the extended regions 13b, 14b, 17b, and 18b, respectively. Each of the connectors 330 includes a cable 331 and a circular conical coil spring-shaped contact terminal 332.

As shown in FIGS. 18 through 21, the contact terminal 332 of the connector 330 makes contact with the extended region 14b that is wrapped around to the rear surface side of the first housing 310, which electrically connects the contact terminal 332 and the extended region 14b. This conduction structure is the same for the electrode 13, the electrode 17, and the electrode 18.

Actions and Effects

The same actions and effects as the body impedance measurement apparatus 100 according to the aforementioned first embodiment can be achieved by the body impedance measurement apparatus 300 according to the present embodiment. In addition, because the same membrane-type electrodes as in the body impedance measurement apparatus 100 according to the first embodiment are used for the electrodes 13, 14, 17, and 18, the extended regions 13b, 14b, 17b, and 18b can be formed with ease, and as described in the present embodiment, a structure in which the extended regions 13b, 14b, 17b, and 18b are wrapped around to the rear surface side using the peripheral edges of the first housing 310 can be employed.

### Fourth Embodiment

### Body Impedance Measurement Apparatus 400

Next, a body impedance measurement apparatus 400 according to a fourth embodiment will be described with reference to FIGS. 22 through 28. The body impedance measurement apparatuses 100 through 300 according to the first through third embodiments described above measure the body impedance of the user by the user standing upon the body impedance measurement apparatus as shown in FIG. 3; however, the body impedance measurement apparatus 400 according to the present embodiment measures the body impedance of the user by using both hands of the user.

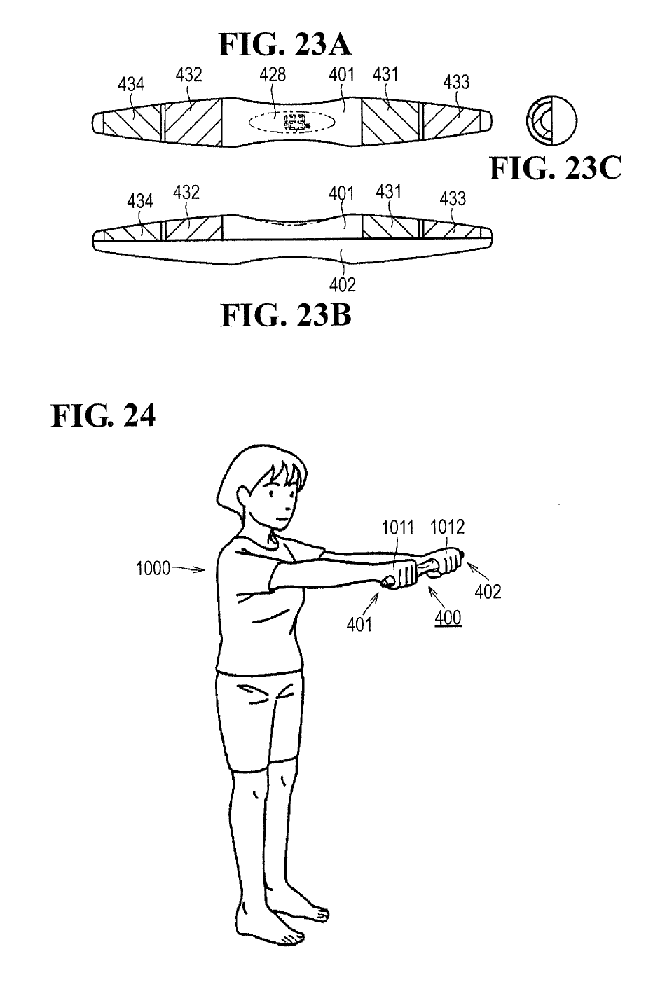

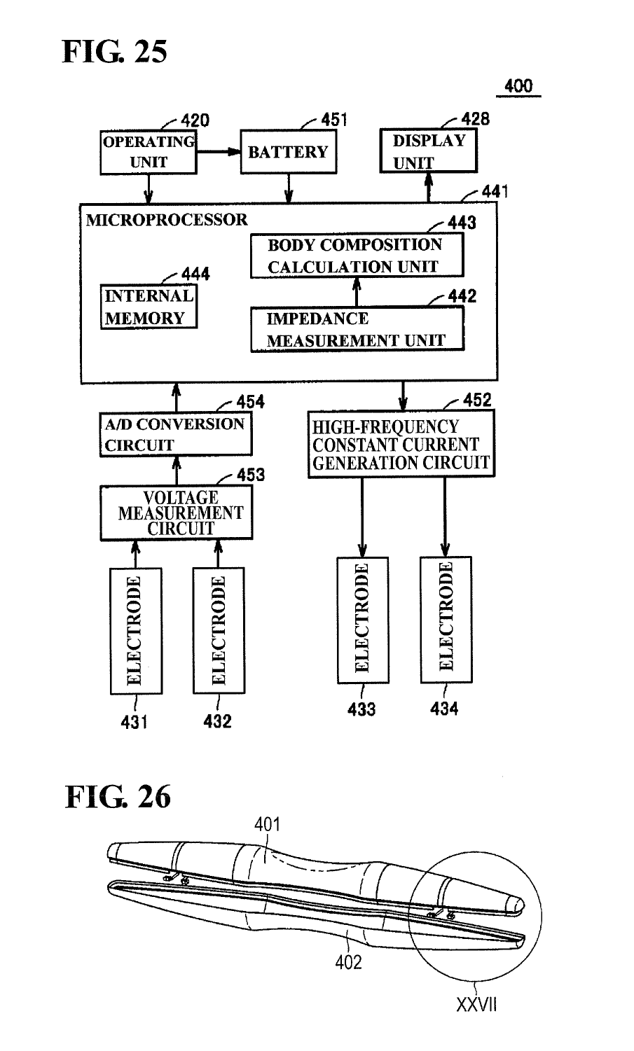

First, the general configuration of the body impedance measurement apparatus 400 will be described with reference to FIGS. 22 through 25. Note that FIG. 22 is a first perspective view illustrating the external configuration of the body impedance measurement apparatus 400, FIGS. 23A, 23B, and 23C are a plan view, a front view, and a right-side view, respectively, illustrating the external configuration of the body impedance measurement apparatus 400, FIG. 24 is a diagram illustrating a measurement posture taken when a user uses the body impedance measurement apparatus 400, FIG. 25 is a block diagram of the body impedance measurement apparatus 400, and FIG. 26 is a second perspective view illustrating the external configuration of the body impedance measurement apparatus 400.

The body impedance measurement apparatus 400 has an overall essentially cylindrical shape, and includes a first housing 401 located on a top surface side (the side on which a display unit 428 (mentioned later) is provided) and a second housing 402 located on a bottom surface side. When viewed from above, the central region of the first housing 401 and the second housing 402 is slightly narrowed, and both ends have essentially conical shapes in which the outer diameters gradually narrow toward those ends.

A right-hand grip portion 411 for gripping with the right hand is provided on the right end thereof, and a left-hand grip portion 412 for gripping with the left hand is provided on the left end thereof. The display unit 428 is provided in the central region of the first housing 401. For example, a liquid-crystal display device (LCD) or the like is used for the display unit 428.

Electrodes 431, 432, 433, and 434 are provided in the hatched regions shown in FIGS. 23A, 23B, and 23C. The electrodes 431 and 433 are provided in predetermined locations on the outer surface of the right-hand grip portion 411. Of the electrodes 431 and 433, the electrode 431 located toward the center of the body impedance measurement apparatus 400 is an electrode for measuring a voltage during impedance measurement, whereas the electrode 433 located toward the right end of the body impedance measurement apparatus 400 is an electrode for applying a current during impedance measurement. The electrodes 431 and 433 make contact with the palm of the user's right hand.

The electrodes 432 and 434 are provided in predetermined locations on the outer surface of the left-hand grip portion 412. Of the electrodes 432 and 434, the electrode 432 located toward the center of the body impedance measurement apparatus 400 is an electrode for measuring a voltage during impedance measurement, whereas the electrode 434 located toward the left end of the body impedance measurement apparatus 400 is an electrode for applying a current during impedance measurement. The electrodes 432 and 434 make contact with the palm of the user's left hand.

Note that the same type of membrane-type electrodes as those used in the body impedance measurement apparatus 100 according to the first embodiment are used as the electrodes 431, 432, 433, and 434 in the present embodiment. In addition, the same material as that used in the body impedance measurement apparatus 100 according to the first embodiment is used in the first housing 401 and the second housing 402.

As shown in FIG. 24, when the body impedance measurement apparatus 400 is being used, the user 1000 grips the right-hand grip portion 411 of the body impedance measurement apparatus 400 with his/her right hand 1011 and the left-hand grip portion 412 of the body impedance measurement apparatus 400 with his/her left hand 1012 while standing erect. At this time, the user straightens both elbows and holds both arms out to approximately the same height at his/her shoulders so that the body impedance measurement apparatus is positioned in front of his/her body, forming essentially a right angle between his/her arms and torso.

As shown in the block diagram in FIG. 25, in addition to the electrodes 431 through 434, the display unit 428, an operating unit 420, and a battery 451, the body impedance measurement apparatus 400 according to the present embodiment includes: a microcomputer 441 for carrying out the overall control of the body impedance measurement apparatus, processes such as various types of computations, and so on; a high-frequency constant current generation circuit 452 that generates a high-frequency constant current of a predetermined frequency; a voltage measurement circuit 453 that measures voltage information obtained by the electrodes 431 and 432 used for voltage measurement; and an A/D (analog/digital) conversion circuit 454 for converting the voltage information obtained from the voltage measurement circuit 453 from an analog signal into a digital signal.

Meanwhile, the microcomputer 441 includes: an impedance measurement unit 442 that measures a body impedance based on the voltage information that has been converted to a digital signal; a body composition calculation unit 443 that calculates a body composition by carrying out computational processes on the obtained impedance; and an internal memory 444, employing an EEPROM (Electrically Erasable Programmable Read-Only Memory) or the like, for storing various types of control programs and the like.

Note that fat mass, fat-free mass, muscle mass, bone mass, body fat percentage, muscle percentage, visceral fat levels, and so on can be given as examples of body compositions that can be measured by the body impedance measurement apparatus 400 according to the present embodiment. These body compositions are all calculated by the body composition calculation unit 443 through known methods based on body impedance values obtained by the stated impedance measurement unit 442 and personal data of the user such as his/her height, weight, age, sex, and so on recorded in the internal memory.

Conduction Structure

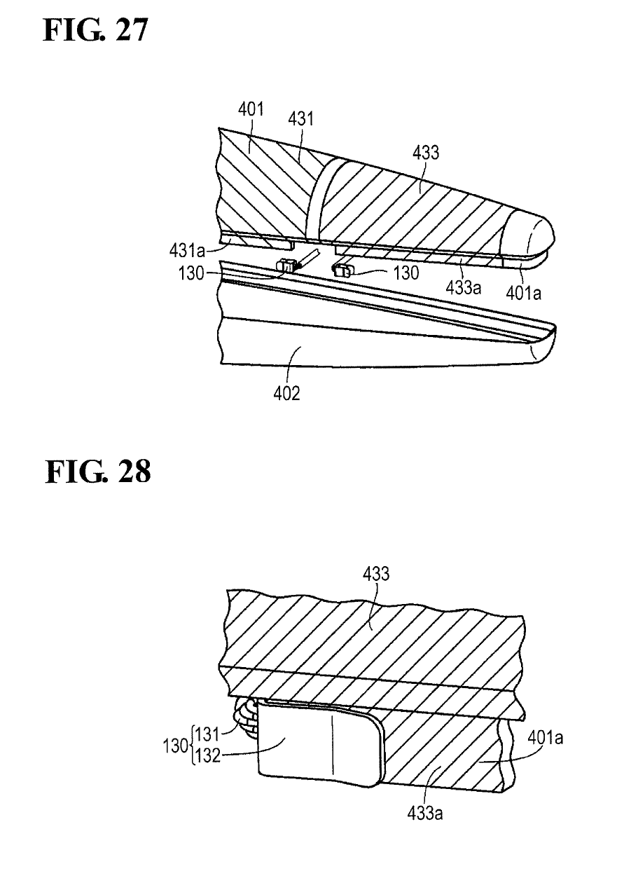

Next, a conduction structure between an electronic component (not shown) housed between the first housing 401 and the second housing 402 and the electrodes 431, 432, 433, and 434 provided on the surface of the first housing 401 will be described with reference to FIGS. 26 through 28. Note that FIG. 26 is a second perspective view illustrating the external configuration of the body impedance measurement apparatus 400, FIG. 27 is an enlarged partial perspective view illustrating the region enclosed by XXVII in FIG. 26, and FIG. 28 is an enlarged partial view illustrating the conduction structure.

As shown in FIG. 27, a rib 401a that extends toward the second housing 402 from a position that is recessed inward is provided on the side of the first housing 401 that faces the second housing 402. In addition, extended regions 431a and 433a that wrap around to the surface of the rib 401a in the first housing 401 are provided in the electrodes 431 and 433.

The electronic component (not shown) is provided with connectors 130 for conductivity with the extended regions 431a and 433a. Each of the connectors 130 includes a cable 131 and a clip 132.

As shown in FIG. 28, the clip 132 of the connector 130 fits around the rib 401a of the first housing 401 on the rear surface side of the first housing 401, thus electrically connecting the clip 132 and the extended region 433a of the electrode 433. This conduction structure is the same for the electrode 431, the electrode 432, and the electrode 434.

Actions and Effects

The same actions and effects as the body impedance measurement apparatus 100 according to the aforementioned first embodiment can be achieved by the body impedance measurement apparatus 400 according to the present embodiment. In addition, because the same membrane-type electrodes as in the body impedance measurement apparatus 100 according to the first embodiment are used for the electrodes 431, 432, 433, and 434, the extended regions can be formed with ease, and thus it is possible to employ a structure in which the extended regions wrap around to the rib provided on the rear surface side of the first housing 401, as described in the present embodiment.

Note that it is also possible to combine the body impedance measurement apparatuses described in the aforementioned first through third embodiments with the body impedance measurement apparatus described in the aforementioned fourth embodiment as appropriate. Furthermore, although the various embodiments describe cases in which a resin material is used as the material of the housing on which the membrane-type electrodes are provided in order to enable the easy formation of curved surfaces, it is also possible to use glass, wood, or the like as the material for the housing on which the membrane-type electrodes are provided instead of a resin material and form the surface thereof in a curved shape, and then add the membrane-type electrodes to that surface.

The foregoing has described exemplary embodiments of the present invention, but it should be noted that the embodiments disclosed above are to be understood as being in all ways exemplary and in no way limiting. The scope of the present invention is defined by the scope of the appended claims, and all changes that fall within the same essential spirit as the scope of the claims are intended to be included therein as well.

### REFERENCE SIGNS LIST

10, 441 microcomputer
13b, 14b, 17b, 18b extended region
13, 14, 17, 18, 431, 432, 433, 434 electrode
13a, 14a, 17a, 18a extended region
20 display unit
30 operating unit
31 power source unit
32 body weight measurement unit
33 external memory
41, 452 high-frequency constant current generation circuit
44 input switching circuit
45, 454 A/D (analog/digital) conversion circuit
100, 200, 300, 400 body impedance measurement apparatus
101, 442 impedance measurement unit
102 resistivity calculation unit
103, 443 body composition calculation unit
110, 210, 310, 401 first housing
111, 211 cap
133, 444 internal memory
112, 212 recessed region
113, 114, 213 protruding region
113s, 114s slit
120, 220, 320, 402 second housing
130, 330 connector
131, 331 cable
132 clip
140 electronic component
141 plate
150 screw
332 contact terminal
401a rib
411 right-hand grip portion
412 left-hand grip portion
428 display unit
431a, 433a extended region
453 voltage measurement circuit
1000 user
1011 right hand
1012 left hand
1013 left foot
1014 right foot

1. A body impedance measurement apparatus that measures the impedance of a body, the apparatus comprising:
a housing in which an electronic component is contained; and
an electrode, provided on a surface of the housing, that makes contact with the body when measuring the impedance of the body,
wherein a base portion of the housing on which the electrode is provided is a resin molded component;
the electrode is a membrane-type electrode
the membrane-type electrode is formed on a film;
the housing includes a first housing and a second housing;
the membrane-type electrode is provided on a surface of the first housing;
the membrane-type electrode includes an extended region that wraps around to the rear surface side of the first housing; and
the extended region is conductive, at the rear surface side of the first housing, with the electronic component contained within the housing.

2. The body impedance measurement apparatus according to claim 1, wherein the film on which the membrane-type electrode is formed is integrated with the surface of the housing through insert molding when the resin of the housing is molded.

3. The body impedance measurement apparatus according to claim 2, wherein the membrane-type electrode is a transparent conductive membrane or a web-form conductive membrane.

4. The body impedance measurement apparatus according to claim 1, wherein the surface of the housing on which the membrane-type electrode is provided has a curved surface portion.
